(19) United States
(12) Patent Application Publication
(10) Pub. No.: US 2019/0025279 A1
(43) Pub. Date: Jan. 24, 2019

(54) TESTING INSTRUMENT WITH QUALITY CONTROL DETERMINATION AND A METHOD THEREOF

(71) Applicant: Chang Gung Medical Technology Co., Ltd., Taipei City (TW)

(72) Inventor: MING-CHEN HSIEH, TAIPEI CITY (TW)

(21) Appl. No.: 15/658,113

(22) Filed: Jul. 24, 2017

(51) Int. Cl. G01N 33/487; G01N 35/00; G01N 33/49

(52) U.S. Cl. CPC G01N 33/48771; G01N 35/00623; G01N 2035/00752; G01N 33/49; G01N 35/00732

(57) ABSTRACT

This disclosure is related to a testing instrument with quality control determination and a method thereof. The testing instrument includes a housing, a monitor, a scanning key, a scanning area, a strip slot and a control circuit. The test instrument has a nursing staff ID table, a strip corresponding table and a quality control testing performance table which are all embedded in the control circuit. The three tables correspond with a step of determining whether the operator matches one of the members listed on the nursing staff ID table, and a step of performing the quality control testing to achieve the automatic determination of whether or not a quality control test has been performed and whether or not the quality control test is passed.

### BACKGROUND OF THE INVENTION

1. Field of the Invention

The present invention relates to a testing instrument and a method thereof; in particular, to a testing instrument with quality control determination and a method thereof.

### 2. Description of Related Art

In the prior art, Point-of-Care Testing (POCT) instruments are very useful for nursing staff to do the clinical care, whereas it is difficult to monitor the testing quality of the instrument, and inaccurate tests would occur frequently. Therefore, the nursing staff needs to inspect the testing quality. However, after working long hours, the nursing staff may forget to inspect the testing quality, or forget whether the quality control test has been done for the day. Another situation that may occur is that even if the quality control test has been carried out, it may be that no one knows whether the previous quality control test has passed the testing standard. Therefore, the Point-of-Care Testing (POCT) instrument is still susceptible to certain incidents. More seriously, inaccurate measurements may cause delays in seeking treatment, leading to unnecessary patient deaths. Consequently, there is still room for improvements in conventional testing instruments.

### SUMMARY OF THE INVENTION

An object of the present invention is to provide a testing instrument with quality control determination and a method thereof. By executing automatic determination and comparison functions in the steps of the quality control test, the screen of the monitor will be automatically switched to the next screen. The operator does not need to confirm whether or not the quality control test has been completed or selectively switch to a desired mode from various modes (such as to switch to the quality control mode or the patient measuring mode), so that the conventional technology and the quality control of medical testing can be effectively improved. In addition, the testing instrument with quality control determination and the method thereof are adapted to various existing Point-of-Care Testing (POCT) instruments.

In order to achieve the aforementioned objects, according to an embodiment of the present invention, a testing instrument with quality control determination, working with a test strip bottle and at least one quality control liquid for executing the quality control test, includes: a housing; a monitor disposed on the top surface of the housing; a scanning button disposed below the monitor; a scanning area disposed on the lower side of the housing, used for scanning a barcode; and a strip slot disposed on one side of the housing; wherein a control circuit is disposed in the housing, the control circuit is coupled to the monitor, the scanning button, the scanning area and the strip slot, used for controlling the operation of the testing instrument and comparing various quality control tests, and determining whether the quality control test is passed; wherein the control circuit stores a nursing staff ID table, a strip corresponding table and a quality control testing performance table as the basis for the comparison of various quality control tests.

In order to achieve the aforementioned objects, according to another embodiment of the present invention, a testing method applied to the testing instrument with quality control determination, wherein the testing method also works with the strip bottle and the quality control liquid, includes: turning on and scanning a barcode of a staff ID; determining whether the staff ID matches that of a member of the hospital nursing staff; determining whether or not a quality control test has been performed for the day; executing the quality control test; determining whether or not the quality control test is passed; and if the quality control test is passed, executing the measurement of the patient.

In order to further the understanding regarding the present invention, the following embodiments are provided along with illustrations to facilitate the disclosure of the present invention.

### BRIEF DESCRIPTION OF THE DRAWINGS

FIG. 1 shows a schematic 3-dimensional diagram according to one embodiment of the present invention;

FIG. 2 shows another schematic 3-dimensional diagram according to one embodiment of the present invention;

FIG. 3 shows a block diagram of the connection of the control circuit according to one embodiment of the present invention;

FIG. 4A shows a schematic diagram of the nursing staff ID table according to one embodiment of the present invention;

FIG. 4B shows a schematic diagram of the strip corresponding table according to one embodiment of the present invention;

FIG. 4C shows a schematic diagram of the quality control testing performance table according to one embodiment of the present invention;

FIG. 5 shows a flowchart according to one embodiment of the present invention;

FIG. 6 shows a flowchart of a testing method of the quality control determination according to one embodiment of the present invention;

FIG. 7 shows a flowchart of a testing method of the quality control determination according to another embodiment of the present invention;

FIG. 8A shows a schematic diagram of a screen of the testing instrument of the present invention;

FIG. 8B shows a schematic diagram of another screen of the testing instrument of the present invention;

FIG. 8C shows a schematic diagram of another screen of the testing instrument of the present invention;

FIG. 8D shows a schematic diagram of another screen of the testing instrument of the present invention;

FIG. 8E shows a schematic diagram of another screen of the testing instrument of the present invention;

FIG. 8F shows a schematic diagram of another screen of the testing instrument of the present invention;

FIG. 8G shows a schematic diagram of another screen of the testing instrument of the present invention; and

FIG. 8H shows a schematic diagram of another screen of the testing instrument of the present invention.

### DETAILED DESCRIPTION OF THE PREFERRED EMBODIMENTS

The aforementioned illustrations and following detailed descriptions are exemplary for the purpose of further explaining the scope of the present invention. Other objectives and advantages related to the present invention will be illustrated in the subsequent descriptions and appended drawings.

An object of the present invention is to provide a testing instrument with quality control determination and a method thereof. By executing automatic determination and comparison functions in the steps of the quality control test, the screen of the monitor will be automatically switched to the next screen. The operator does not need to confirm whether or not the quality control test has been completed or selectively switch to a desired mode from various modes (such as to switch to the quality control mode or the patient measuring mode).

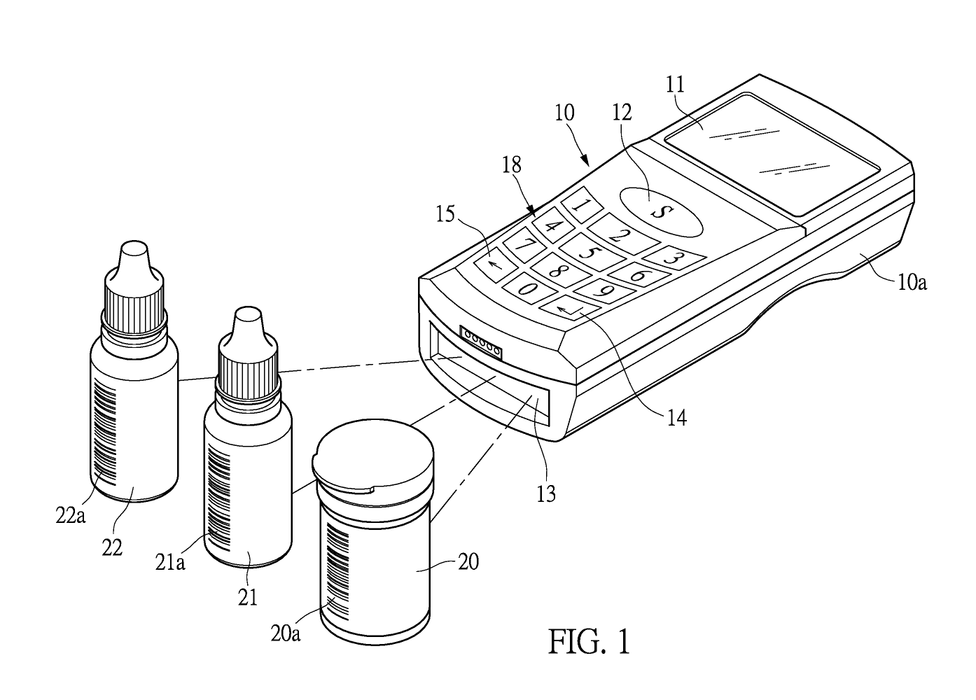

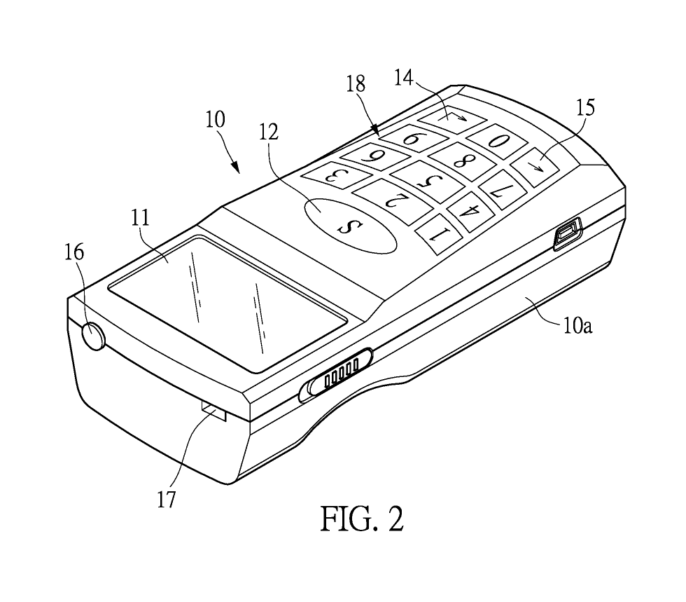

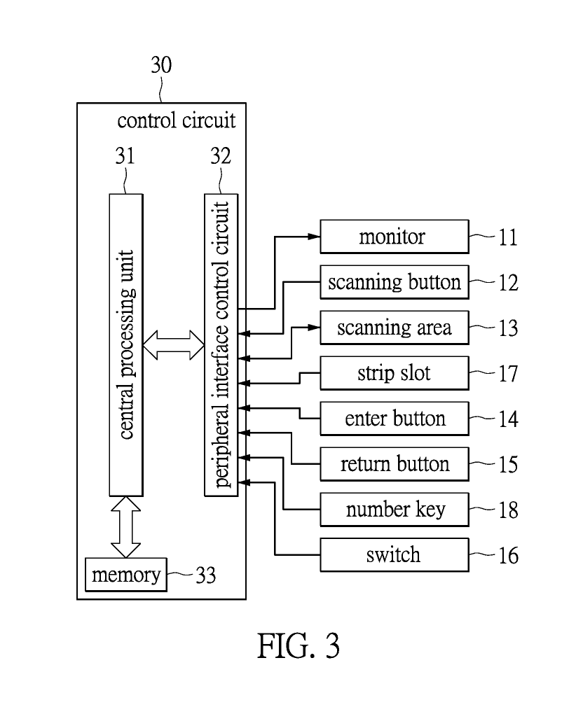

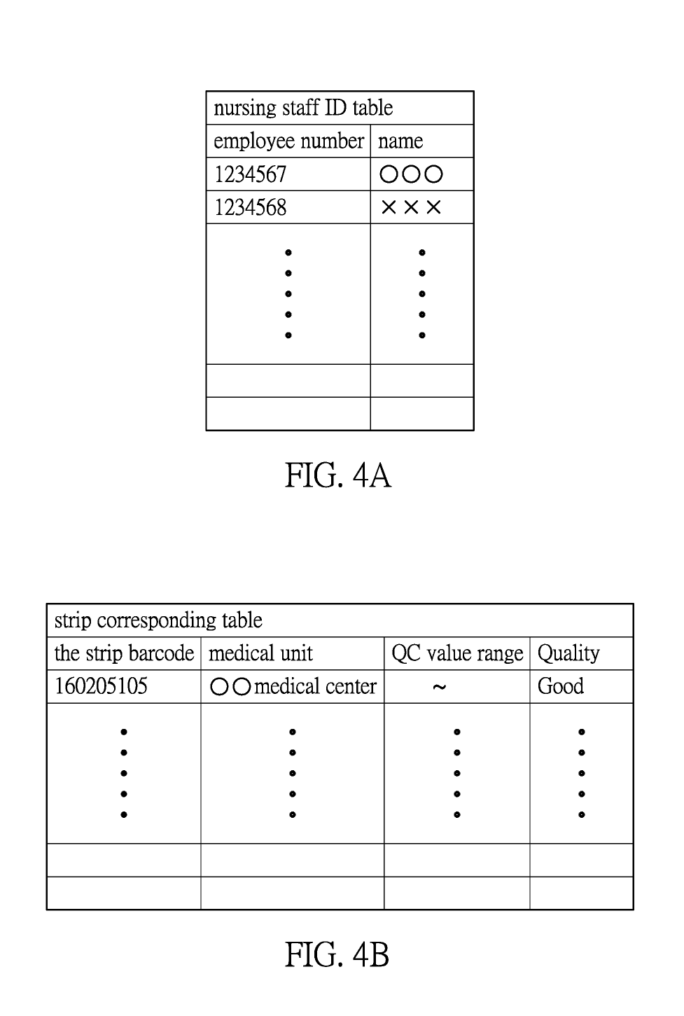

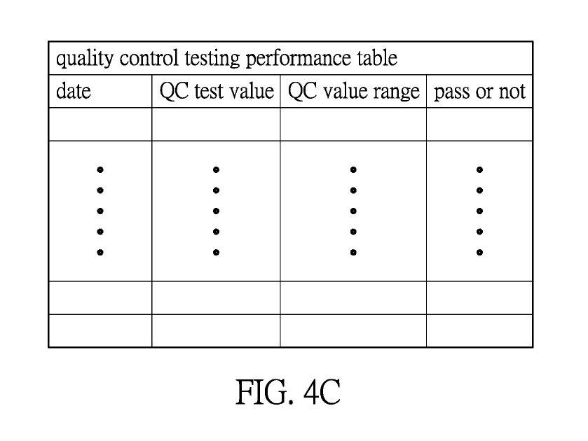

Referring to FIG. 1 and FIG. 2, a testing instrument 10 with quality control determination working with a strip bottle 20 and a first quality control liquid 21 and/or a second quality control liquid 22 for executing the quality control test (the steps of operation are shown in FIG. 4). The testing instrument 10 includes a housing 10a, a monitor 11, a scanning button 12, a scanning area 13, an enter button 14, a return button 15, a switch 16, a strip slot 17 and a set of number keys 18. The monitor 11 is disposed on the top surface of the housing 10a, the scanning button 12 is disposed below the monitor 11, and the scanning area 13 is disposed on the lower side of the housing 10a, wherein the scanning area 13 is used for scanning a barcode. The switch 16 is disposed on one side of the housing 10a for turning on or turning off the testing instrument 10, and the strip slot 17 is disposed on one side of the housing 10a. A control circuit 30 (as shown in FIG. 3) is disposed inside the housing 10a. The control circuit 30 is coupled to the monitor 11, the scanning button 12, the scanning area 13 and the strip slot 17, and is used for controlling the operation of the testing instrument 10 and comparing various quality control tests and determining whether the quality control test is passed. The control circuit 30 stores a nursing staff ID table (as shown in FIG. 4A), a strip corresponding table (as shown in FIG. 4B), and a quality control testing performance table (as shown in FIG. 4C) as the basis for the comparison of various quality control tests.

In FIG. 1, the strip bottle 20 has a strip bottle barcode 20a, the first quality control liquid 21 has a first quality control liquid barcode 21a, and the second quality control liquid 22 has a second quality control liquid barcode 22a. In FIG. 1 and FIG. 2, the enter button 14, the return button 15, and the number keys 18 are disposed on the top surface of the housing 10a, so that the operator or the nursing staff can conveniently operate the testing instrument 10 by using the enter button 14, the return button 15, and the number keys 18. In practice, only the enter button 14 and the return button 15 can be disposed on the top surface of the housing 10a, so that the design of the testing instrument 10 can be simplified and the cost of setting the number keys 18 can be saved.

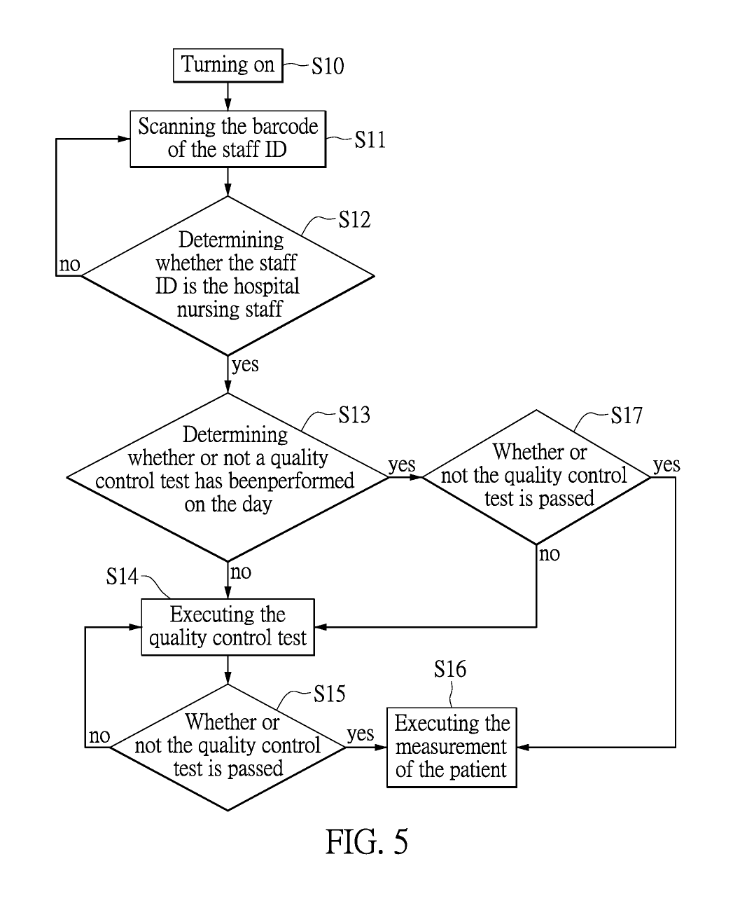

FIG. 3 shows a block diagram of the connection to peripheral components of the control circuit 30. The control circuit 30 includes a central processing unit 31, a peripheral interface control circuit 32 and a memory 33. The central processing unit 31 is used for executing every control action of the tests of operating the testing instrument 10, such as determining whether the operator is a member of the hospital nursing staff. For example, the control of the steps shown in FIG. 5 to FIG. 7 is executed by the central processing unit 31. The peripheral interface control circuit 32 is coupled to the central processing unit 31 and also coupled to the monitor 11, the scanning button 12, the scanning area 13, the strip slot 17, the enter button 14, the return button 15, the number keys 18 and the switch 16. The central processing unit 31 of the control circuit 30 executes the related control of the testing instrument 10 through the connection of the peripheral interface control circuit 32 to the monitor 11, the scanning button 12, the scanning area 13, the strip slot 17, the enter button 14, the return button 15, the number keys 18 and the switch 16. The memory 33 is coupled to the central processing unit 31 for storing the nursing staff ID table as shown in FIG. 4A, the strip corresponding table as shown in FIG. 4B and the quality control testing performance table as shown in FIG. 4C as the basis for the comparison and determination of the quality control tests.

In FIG. 4A, the nursing staff ID table can include two fields, being the employee number and employee name for the sake of clarity, to which the present invention is not limited. In practice, the nursing staff ID table is not limited to the two fields. For example, a third field can be added, and the third field can be used to identify the department or section of the nursing staff to serve as a further confirmation of the nursing staff. In FIG. 4B, the strip corresponding table includes three fields of the strip barcode, the medical unit and the QC value range and quality to show the source identification of the strip to be used by the operator, and to provide the determination of whether the strip to be used for the test is certified by a qualified medical unit. In practice, the strip corresponding table can add a fifth field to identify information relating to shelf life. In FIG. 4C, the quality control testing performance table includes the four fields of date, the QC test value, the QC value range, and pass or fail, in which “QC” is the abbreviated form of Quality Control. The QC test value indicates the quality control test value under test. The QC value range is the predetermined standard value of the QC test, and is used to compare to the QC test value so as to determine whether the quality control test is passed.

FIG. 5 is related to the testing method of the present invention applied to the testing instrument 10 with quality control determination, where the testing method also works with the strip bottle 20 and at least one quality control liquid, for example, the first quality control liquid 21 and/or the second quality control liquid 22. In FIG. 5, the testing method includes steps S10-S17. In step S10: turning on the testing instrument 10. In step S11: scanning the barcode of the staff ID. In step S12: determining whether the staff ID matches that of a member of the hospital nursing staff. If not, the process returns to step S11; if yes, the process proceeds to step S13: determining whether or not a quality control test has been performed for the day. If not, the process proceeds to step S14: executing the quality control test. If the quality control test has been performed, the process proceeds to step S17: whether or not the quality control test is passed. If it is passed, step S16 is performed: executing the measurement of the patient. If it is not passed, the process goes back to step S14: executing the quality control test. After step S14 is performed, the process proceeds to step S15: whether or not the quality control test is passed. If it is passed, the process proceeds to step S16. If it is not passed, the process returns to step S14. The processes mentioned above complete the testing method of the testing instrument with quality control determination.

The step of determining whether the staff ID matches that of a member of the hospital nursing staff in step S12 is to compare the data from scanning the barcode of the staff ID to the nursing staff ID table stored in the control circuit 30 of the testing instrument 10. If the comparison shows a match, the staff ID is determined to be a member of the hospital nursing staff. The steps of determining whether or not the quality control test is passed in step S14 can be divided into two test parts. One part is to compare the data of a strip (taken from the strip bottle 20) obtained from the quality control test to the strip corresponding table stored in the control circuit 30 of the testing instrument 10. If the comparison shows a match, the strip passes the test. The other part is to compare the data of the quality control liquid (taken from the first quality control liquid 21 or the second quality control liquid 22) obtained from the quality control test to the quality control testing performance table stored in the control circuit 30 of the testing instrument 10. If the comparison shows a match, the quality control liquid passes the test.

According to another embodiment of the present invention, the steps regarding whether or not the quality control test is passed in step S15 further include: when the operator (i.e., the hospital nursing staff) finds an abnormal condition or the quality control test does not match the pre-stored standard, the abnormal value measured by the testing instrument 10 is uploaded to a remote receiver, and the receiver automatically determines that the value is abnormal. After that, the receiver sends a message to the operator's upper manager, for example, the supervisor of the operator. Therefore, this embodiment of the present invention provides the function of monitoring and management, and the operator continues the operation of confirming the relevant tests. After the quality control test is completed and the test is passed, the new values are transmitted to the receiver, and then to a supervisor for confirmation so as to achieve the purpose of monitoring and management.

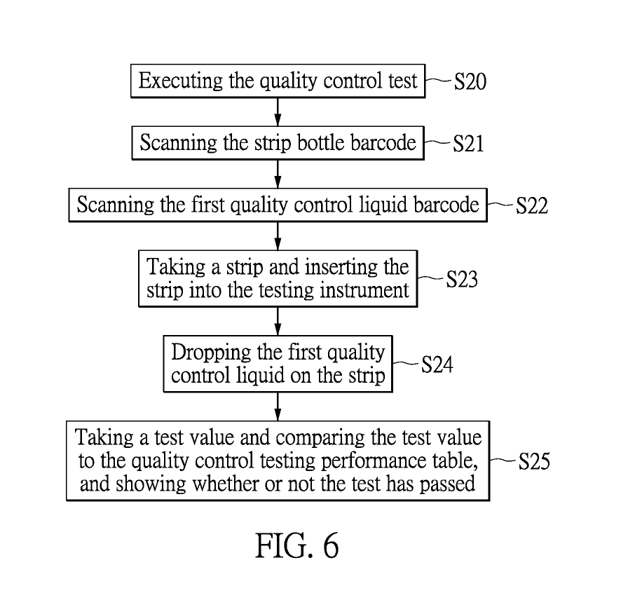

The quality control test for step S14 is further described in FIG. 6, including step S20: executing the quality control test. The process then proceeds to step S21: scanning the strip bottle barcode (the strip bottle barcode 20a). In practice, the operator presses the scanning button 12 to make the scanning area 13 emit red light, and then places the strip bottle barcode 20a in the red light. The testing instrument 10 reads the strip bottle barcode 20a and displays it on the monitor 11.The scanning of strip bottle barcode of step S21 is then complete. Step S22 is performed to scan the first quality control liquid barcode. Likewise, in this step, the operator presses the scanning button 12 to make the scanning area 13 emit red light, and then places the first quality control liquid barcode 21a in the red light. The testing instrument 10 reads the first quality control liquid barcode 21a and displays it on the monitor 11.The scanning of the first quality control liquid barcode of step S22 is then complete. Step S23 is performed: taking a strip and inserting the strip into the testing instrument 10. Step S24 is performed: dropping the first quality control liquid on the strip. Step S25 is performed: taking the test value and comparing it to the quality control testing performance table, and showing whether or not the test has been passed. After that, the steps of the testing method of the quality control test are complete. The scanning of the strip bottle barcode of step S21 is to compare the scanning result to the strip corresponding table stored in the memory 33 of the testing instrument 10.If the comparison does not show a match, the monitor displays that the test was not passed.

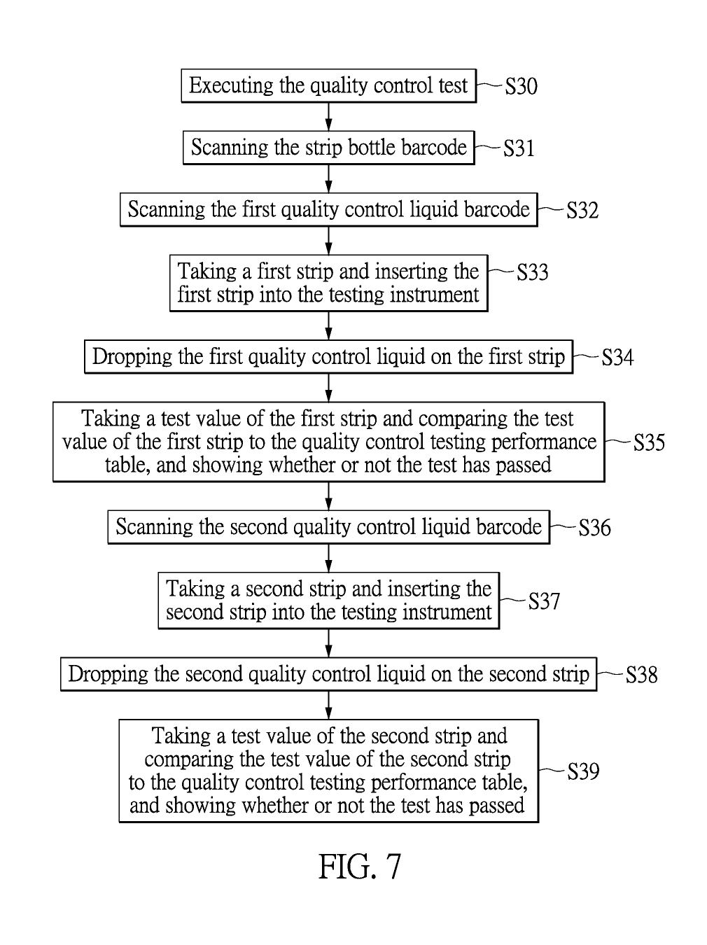

FIG. 7 shows another embodiment of the quality control test of step S14. The difference between the embodiment of FIG. 6 and that of FIG. 7 is that the quality control liquid used in FIG. 7 includes the first quality control liquid 21 and the second quality control liquid 22. In practice, the test value of the first quality control liquid 21 may be used as an upper limit of the quality control test and the second quality control liquid 22 may be used as a lower limit of the quality control test to form a test value range for the testing of the testing instrument 10. In step S30: executing the quality control test. Step S31: scanning the strip bottle barcode. Step S32 is performed: scanning the first quality control liquid barcode. Step S33 is performed: taking a first strip and inserting the first strip into the testing instrument 10. Step S34 is performed: dropping the first quality control liquid on the first strip. Step S35 is performed: taking the test value of the first strip and comparing it to the quality control testing performance table, and showing whether or not the test is passed. Step S36 is performed: scanning the second quality control liquid barcode. Step S37 is performed: taking a second strip and inserting the second strip into the testing instrument 10. Step S38 is performed: dropping the second quality control liquid on the second strip. Step S39 is performed: taking the test value of the second strip and comparing it to the quality control testing performance table, and showing whether or not the test is passed. After that, another embodiment of the steps of the testing method of the quality control test is completed. The scanning of strip bottle barcode of step S31 is to compare the scanning result to the strip corresponding table stored in the memory 33 of the testing instrument 10. If the comparison does not show a match, the monitor displays that the test was not passed.

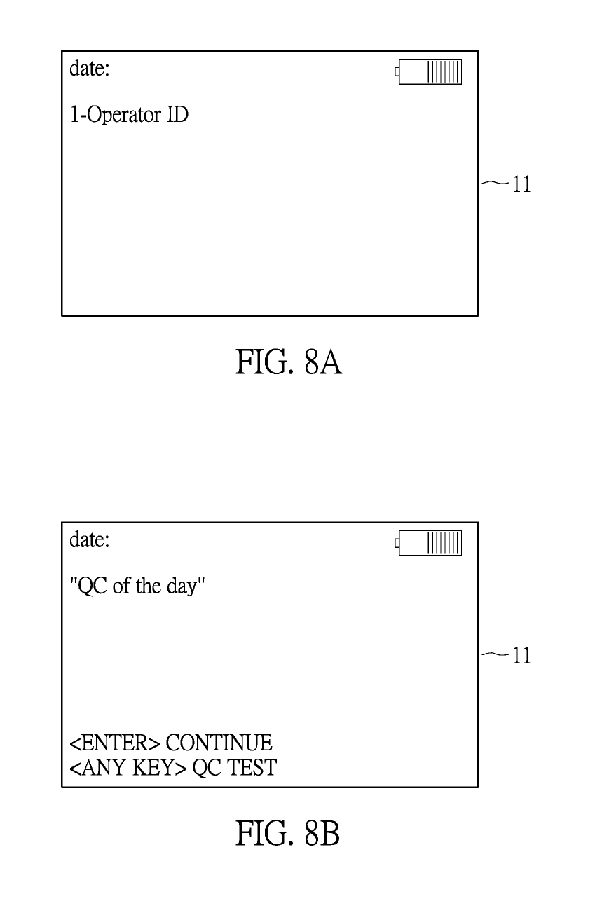

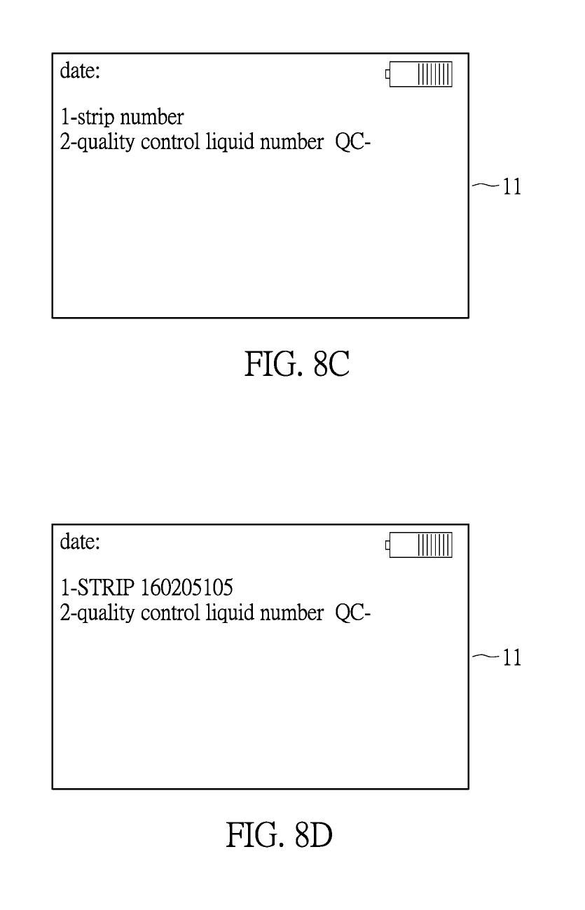

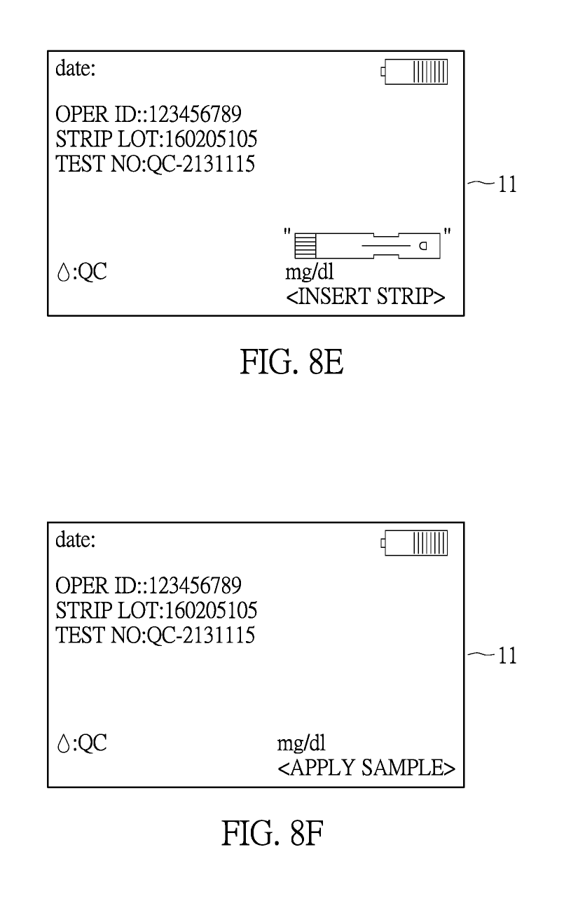

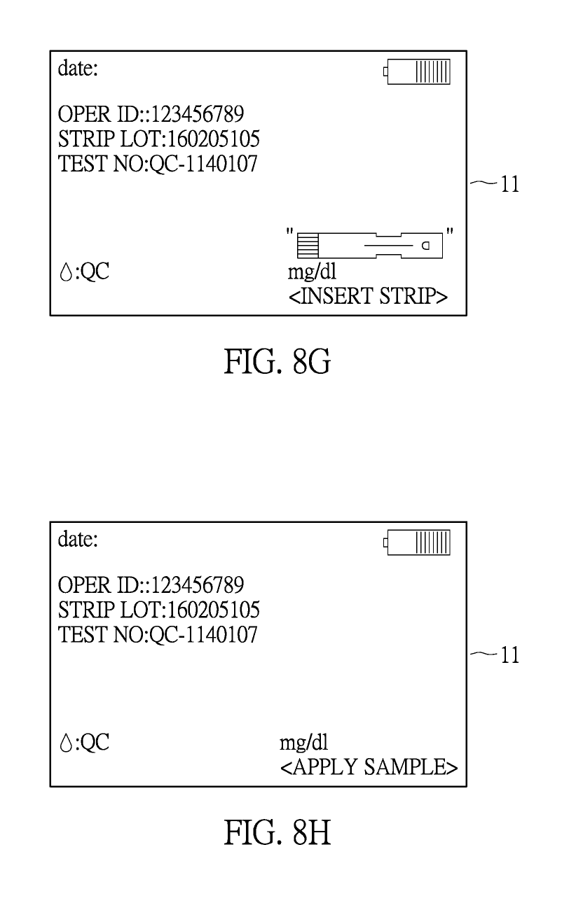

FIGS. 8A to 8H show the screens displayed on the monitor 11 according to the steps in FIGS. 5, 6 and 7. FIG. 8A is the display screen of monitor 11 after step S10 is executed in order to prompt the operator to scan the ID of the operator. The operator presses the scanning button 12 to align the scanning area 13 with the barcode of the operator's identification card. The control circuit 30 of the testing instrument 10 automatically compares the scanning result to the nursing staff ID table. If it is determined that the nursing staff is listed in the table, the employee ID and the name of the nursing staff are displayed on the monitor 11.

FIG. 8B shows the execution of step S13: determining whether or not a quality control test has been performed for the day. If the quality control test has not been performed yet, the monitor 11 displays the screen as shown in FIG. 8B, and then switches to the next screen as shown in FIG. 8C. FIG. 8C corresponds to the screen of executing the quality control tests described in steps S14, S20 or S30. That is, the monitor 11 displays “1-strip number” and “2-quality control liquid number”. At this time, the operator needs to press the scanning button 12 to align the scanning area 13 with the strip bottle barcode 20a for the scanning. Corresponding to step S21, the control circuit 30 of the testing instrument 10 automatically compares the strip bottle to the strip corresponding table. If the strip bottle is determined to be qualified, it is indicated that there is no problem with the strip, and the test is passed. The monitor 11 then displays a batch number corresponding to the strip bottle as shown in FIG. 8D. The screen in FIG. 8D is also prompting the operator to execute the quality test for the quality control liquid. Corresponding to step S22, the operator needs to press the scanning button 12 to align the scanning area 13 with the first quality control liquid barcode 21a for the scanning. The control circuit 30 of the testing instrument 10 automatically compares the quality control liquid to the quality control testing performance table. If it is determined to be the qualified QC value or within the QC value range, it indicates that there is no problem with the quality control liquid, and the test is passed. The monitor 11 then displays a batch number corresponding to the first quality control liquid barcode 21a as shown in FIG. 8E.

On the other hand, FIG. 8E also prompts the operator to insert the strip in addition to showing the batch number of the first quality control liquid. Corresponding to step S23: taking a strip and inserting the strip into the testing instrument, and step S24: dropping the first quality control liquid on the strip, a display such as that shown in FIG. 8F appears. The space before the mg/dL shows the value as the basis for determining whether or not the test was passed. In addition, the display shown in FIG. 8G corresponds to that in FIG. 8E, but is for expressing the batch number of the second quality control liquid 22 displayed after scanning the second quality control liquid barcode 22a corresponding to step S36. FIG. 8G also prompts the operator to insert the second strip into the testing instrument 10. Corresponding to step S37: taking a second strip and inserting the second strip into the testing instrument 10 and step S38: dropping the second quality control liquid on the second strip, a display such as that shown in FIG. 8H appears. The space before the mg/dL shows the value as a basis for determining whether or not the test is passed.

The aforementioned testing instrument 10 with quality control determination and the method thereof are adapted to Point-of-Care Testing (POCT) instruments for blood glucose testing. Therefore, the present invention can be applied to the existing blood glucose testing devices and is also suitable for blood glucose testing devices of various brands. Further, the testing instrument 10 of the present invention or the testing method thereof can be used in equipment that test medical fluids or drops to improve the accuracy of medical testing and quality of medical care.

In summary, the testing instrument with quality control determination and the testing method thereof according to the present invention has a control circuit 30 disposed in the testing instrument 10. The control circuit 30 stores a nursing staff ID table, a strip corresponding table and a quality control testing performance table as the basis for the comparison of various quality control tests.

The descriptions illustrated supra set forth simply the exemplary embodiments of the present invention; however, the characteristics of the present invention are by no means restricted thereto. All changes, alterations, or modifications conveniently considered by those skilled in the art are deemed to be encompassed within the scope of the present invention delineated by the following claims.

What is claimed is:

1. A testing instrument with quality control determination, working with a strip bottle and at least one quality control liquid for executing a quality control test, comprising:
a housing;
a monitor disposed on the top surface of the housing;
a scanning button disposed below the monitor;
a scanning area disposed on the lower side of the housing, used for scanning a barcode; and
a strip slot disposed on one side of the housing;
wherein a control circuit is disposed in the housing, coupled to the monitor, the scanning button, the scanning area and the strip slot, and used for controlling the operation of the testing instrument, comparing various quality control tests, and determining whether the quality control test is passed;
wherein the control circuit stores a nursing staff ID table, a strip corresponding table and a quality control testing performance table as the basis for the comparison of various quality control tests.

2. The testing instrument with quality control determination according to claim 1, wherein the testing instrument further comprises an enter button and a return button disposed on the top surface of the housing such that a user can conveniently operate the testing instrument;
wherein a switch is disposed on one side of the housing of the testing instrument for turning on and turning off the testing instrument.

3. A testing method applied to the testing instrument with quality control determination according to claim 1, wherein the testing method also works with the strip bottle and the quality control liquid, comprising:
turning on and scanning a barcode of a staff ID;
determining whether the staff ID is matches that of a member of the hospital nursing staff;
determining whether or not a quality control test has been performed for the day;
executing the quality control test;
determining whether or not the quality control test is passed; and
if the quality control test is passed, executing the measurement of the patient.

4. The testing method according to claim 3, wherein the step of determining whether the staff ID is the hospital nursing staff is to compare the data of scanning the barcode of the staff ID to the nursing staff ID table stored in the control circuit of the testing instrument with quality control determination; if a comparison shows a match, the staff ID is determined as matching that of a member of the hospital nursing staff

5. The testing method according to claim 3, after the step of determining whether or not a quality control test has been performed for the day is performed; if the quality control test has been performed, determining whether or not the quality control test is passed; if the quality control test is passed, executing the measurement of the patient; and if the quality control is not passed, executing the quality control test.

6. The testing method according to claim 3, wherein the step of determining whether or not the quality control test is passed is to compare the data of a strip obtained from the quality control test to the strip corresponding table stored in the control circuit of the testing instrument with quality control determination; if a comparison shows a match, the strip passes the test.

7. The testing method according to claim 3, wherein the step of determining whether or not the quality control test is passed is to compare the data of a quality control liquid obtained from the quality control test to the quality control testing performance table stored in the control circuit of the testing instrument with quality control determination; if a comparison shows a match, the quality control liquid passes the test.

8. The testing method according to claim 3, wherein the steps of executing the quality control test comprise:
scanning a strip bottle barcode;
scanning a quality control liquid barcode;
taking a strip and inserting the strip into the testing instrument;
dropping the quality control liquid on the strip; and
taking a test value and comparing the test value to the quality control testing performance table, and showing whether or not the test is passed.

9. The testing method according to claim 8, wherein the steps of executing the quality control test comprise:
scanning the strip bottle barcode;
scanning a first quality control liquid barcode;
taking a first strip and inserting the first strip into the testing instrument;
dropping the first quality control liquid on the first strip;
taking a test value of the first strip and comparing the test value of the first strip to the quality control testing performance table, and showing whether or not the test is passed;
scanning a second quality control liquid barcode;
taking a second strip and inserting the second strip into the testing instrument;
dropping the second quality control liquid on the second strip; and
taking a test value of the second strip and comparing the test value of the second strip to the quality control testing performance table, and showing whether or not the test is passed;
wherein the first quality control liquid is an upper limit of the quality control test, and the second quality control liquid is a lower limit of the quality control test.

10. The testing method according to claim 8, wherein the testing instrument is a Point-of-Care Testing (POCT) instrument for blood glucose testing.

11. The testing method according to claim 9, wherein the testing instrument is a Point-of-Care Testing (POCT) instrument for blood glucose testing.
